(19) United States
(12) Patent Application Publication
(10) Pub. No.: US 2010/0070188 A1
(43) Pub. Date: Mar. 18, 2010

(54) Intelligent medical device system for on-demand diagnostics

(76) Inventor: Neal Solomon, Oakland, CA (US)

Correspondence Address: Neal Solomon, P O Box - 21297, Oakland, CA, 94620, US

(21) Appl. No.: 12/462,780

(22) Filed: Aug. 7, 2009

Related U.S. Application Data
(60) Provisional application No. 61/188,413, filed on Aug. 8, 2008.

(51) Int. Cl. G06F 19/00; C12M 1/34

(52) U.S. Cl. 702/19; 435/287.2; 435/287.1

(57) ABSTRACT

The system describes the diagnostic procedures involving the intelligent medical device (iMD) system. After data is collected from a patient by using cellular and molecular samples, the device detects and analyzes pathology. The system uses lab on a chip

(LOC) data analysis, including microarrays, to analyze DNA mutations and protein anomalies. The medical pathologies are modeled by the iMD computer system and solutions to multi-objective optimization problems are organized using scenario options. The diagnostic functionality of the iMD system also tracks complex regulatory network pathologies.

### CROSS-REFERENCES TO RELATED APPLICATIONS

The present application claims the benefit of priority under 35 U.S.C. §119 from U.S. Provisional Patent Application Ser. No. 61/188,413, filed on Aug. 8, 2008, the disclosure of which is hereby incorporated by reference in their entirety for all purposes.

### FIELD OF THE INVENTION

The invention involves micro electro mechanical systems (MEMS) applied to medical devices and components. The present system includes devices and components for micro total analysis systems (μTAS) and lab on a chip (LOC) apparatuses used in diagnostic aspects of medical intervention.

### BACKGROUND

As scientists discover the mechanics of genetic processes, our understanding of the sources of diseases increases. The benefits of understanding genetic dynamics and proteomics regulatory processes assists in development of a new generation of medical devices able to diagnose, regulate, manage and cure complex diseases. The potential exists to develop personalized drug therapies to target specific genetic pathologies.

Regarding diagnostic systems, MEMS is an umbrella for a class of new medical devices able to identify genetic mutations and proteomic dysfunctions. While largely external in vitro devices, DNA microarrays, RNA microarrays and protein microarrays provide feedback to identify an individual's genetic information. Protein microarrays use antibodies to assess protein functional responses. In addition, whole cell assays test cells with analytes to assess specific responses to chemical inputs. Multi-phenotype cellular arrays are used for bio-sensing of specific inputs in order to study cell functions.

Though DNA, RNA, protein and whole cell arrays have developed separately, a new generation of lab on chip (LOC) and micro-total analysis systems (μTAS) technologies have emerged as well that integrate several functions in a single device. These multi-purpose arrays provide clinical diagnostic data to practitioners.

In addition to these external devices, the evolution of radiological diagnostic tools has provided a revolution to analytical practitioners. In particular, the use of CT, PET and MRI technologies provides detailed data on specific disease progression. In addition to these external radiological diagnostic technologies, the internal sensing “pill” camera records and transmits digital images to substitute for the surgical intervention of exploratory surgery. Finally, the use of implanted sensors assists in the regulation of simple deterministic expert systems.

The convergence of nanotechnology with biology has produced “bionano” devices. In the main, the use of nanotechnology is limited to particles that are targeted to specific tissue in order to identify pathology and, when combined with directed radiation, provide a therapeutic alternative. The advent of self-assembled peptide nano-biomaterials provides interesting opportunities for diagnostics and therapeutics. The use of nano-scale devices, in which collective behaviors are controlled for therapeutic as well as diagnostic modes, provides an advancement of the bionano field.

Regarding therapeutic medical devices and systems, the field has evolved from the development of the hearing aid and the cardiac pace maker. For instance, the implantable brain pacemaker has been developed to regulate epileptic energy pulses and blood glucose monitoring is regulated with an insulin pump. Moreover, implantable pain management devices are used to control chronic pain. Microfluidic devices to target drug delivery, primarily using a deterministic expert system control model, have also been developed. All of these devices are simple single-function mechanisms targeted to a specific disease or disorder.

An emerging scientific field is providing a new set of technologies from bio-inspired computing. Complexity science deals with self-organizing systems that learn in indeterministic environments. The inspiration from the autonomic nervous system and the human immune system provide computing systems that emulate these complex biological processes. Autonomic computing self-diagnoses, self-heals and self-regulates distributed networks. The human immune system provides inspiration for immunocomputing models that emulate protein regulatory network behaviors in order to solve complex optimization problems. Swarm intelligence metaheuristics provides solutions to optimization problems as well. For instance, the ant colony optimization (ACO) metaheuristic provides a model to solve network computing problems. These models share the ability to develop solutions to problems in self-organizing systems, including plasticity behaviors, in indeterministic environments. In effect, these complex computing and control systems learn. So far, these complex computing models have not been applied to medical devices.

The ability to use genetic and proteomic information to solve complex pathologies provides a new generation of opportunities to build medical devices that are customized to each individual's specific disease(s). Our understanding of cancer, for instance, as the combination of multiple genetic mutations, suggests that each disease type is classed into a typology that can be solved with specific targeted therapies. Given this new knowledge, it is logical to build medical devices that are personalized to specific diseases of each individual. In particular, the use of medical devices focused on solving problems involving pathologies associated with cardiovascular, neurological, immunological and endocrinological systems, and with cancer, is a next step.

Each of the prior medical devices has limitations. For the most part, none of the implantable medical devices are “intelligent”. Rather, they are simple deterministic systems. They are also single function devices focused on a specific narrow medical problem. Because they are merely deterministic expert systems, they do not combine diagnostic and therapeutic functionality. In the diagnostic mode, they do not provide sophisticated modeling functions. Further, prior MDs are not networked since they typically involve a single device performing a single function. Finally, these devices are not useful in personalized medicine, which require complex analysis and targeting of individual therapies to unique problem sets.

What is needed? We need active intelligent medical devices that are able to work with other medical devices to solve multiple medical problems. We need complex medical devices that are capable of integrating diagnostics and therapeutics in order to maximize efficiency, to promote early detection and treatment and to modify functionality with feedback mechanisms to solve complex biological optimization problems in biological regulatory networks. The present system develops an intelligent multifunctional medical device system.

Problems that the System Solves

The present system solves a range of problems. How can we develop an intelligent medical device (iMD) that coordinates diagnosis and therapy? How can the iMD coordinate sensors and integrated circuits? How is the processing of chemical and biological fluids administered by using the iMD? How is the implantable iMD coordinated with external computation and modeling? How does the device collect samples and data in real time? How does one integrate multi-functionality into an efficient iMD design? How is the implantable device installed with minimal invasiveness? How are nano-components integrated into the iMD? How does the iMD use sensors and probes for maximum effect? How does the iMD efficiently analyze biological data? How are solutions to complex problems developed and refined in the iMD? How is drug delivery optimized in the iMD? How can we construct customized drugs for therapies to individual patient pathologies? How can an iMD self-organize and adapt to indeterministic environmental conditions? How can multiple iMDs be coordinated, particularly for multiple applications? Solving these problems presents opportunities to develop a new generation of highly effective medical devices.

### SUMMARY OF THE INVENTION

The diagnostic component of the iMD system collects biological samples, such as biomarkers, cell samples, genes and proteins for analysis. The diagnostic module uses a network of probes and sensors to collect data and cell samples. The samples are then analyzed in the on-board lab on a chip (LOC). Specific genetic mutations are assessed, the combinations of which generate specific pathology. The data is then analyzed by on-board computers in conjunction with external computation resources.

The system solves multi-objective optimization problems (MOOPs) by generating analyses and models of diseases. The system builds a model of the disease and generates a set of scenarios within probabilities based on multiple factors. The system offers a range of solutions to the MOOPs for therapeutic intervention.

### Novelties

The ability to integrate LOC functions with analytical functions in one system is novel. The application of advanced metaheuristics to iMDs shows the ability to solve medical diagnostic MOOPs. By computationally modeling complex problems, optimization solutions are provided by iMDs in real time. Further, modeling allows the system to anticipate pathology developmental scenarios that are more easily solved.

### Advantages of the Invention

There are a number of advantages of the present invention. Diagnostic analyses of biomedical problems are performed by the iMDs for rapid, efficient, precise and on-demand therapeutic response. The system automatically assesses chemical biomarkers and cell structure and function and responds to the underlying disease.

The invention allows the integration of diagnostics with therapeutics, thereby increasing the efficiency of the therapeutic modality. The integrated device allows the tracking of therapies by assessing feedback processes in order to more effectively manage complex regulatory networks.

Finally, the ability to provide an early disease detection process in an automated diagnostic system saves time and money.

### DESCRIPTION OF THE INVENTION

(1) Diagnostic Processes of Mapping Pathology using IMDs

Every disease occurs over a multi-phasal process. This pathology evolution process allows multiple symptoms to be tracked and early disease symptoms to be identified.

It is increasingly evident that many diseases have a genetic origin. The combination of genetic mutations that produce dysfunctional proteins create malfunctions in protein regulatory networks that constitute a broad range of specific pathologies. Genetic mutations accumulate over time, which explains why the elderly typically have multiple diseases and why aging is associated with cellular and biomedical system degradation. Tracking genetic disease progression typically begins with a patient's family history. In order, then, to understand specific pathologies, it is necessary to analyze genetic causes of disease as well as the causes of pathology at the cellular level.

The iMD system is used as part of a set of protocols to detect, to collect biological data about and to develop diagnoses for specific pathologies. One fundamental approach to collecting biological data and detecting disease involves using microarrays. Microarrays are devices that test multiple bio-chemicals for specific attributes. Molecular diagnostics such as DNA microarrays, RNA microarrays and protein microarrays test for specific abnormalities in DNA, RNA and proteins, respectively. Whole cell and antigen microarrays use analytes and antibodies, respectively, to test for responses to specific inputs. These molecular diagnostic analyses go beyond, and supplement, traditional radiological imaging tools and techniques.

Historically, most analytical approaches to disease diagnostics use a simple expert system. In this model, information is collected and diagnoses advanced about specific pathological symptoms. These approaches use a straightforward method of assessing and treating the disease symptoms rather than understanding and solving the underlying problem. For this reason, the new generation of genetic and proteomic assessment models brings us to the frontiers of personalized medicine.

(1) Data Collection with IMDs: Biomarker, Protein and Enzyme Detection

The iMD system provides a unique set of methods to diagnose diseases at the cellular and molecular levels. The iMD sends out probes to extract cellular and molecular samples from affected tissues in order to detect protein and enzyme biomarkers. These probes send biological samples to the iMD for analysis. The analysis then further guides later phases of probe direction, detection of disease biomarker attributes and transmission of data. This interactive process is critical to focus on the collection and analysis of cellular and molecular data about complex pathologies.

The system sends probes to specific tissue locations and receives the data back, either from communications from the probe or retrieval of the probe. The lab on a chip (LOC) onboard the iMD then performs an analysis of specific biochemicals using a multi-attribute microarray, seeking to detect molecular biomarkers or a combination of genetic SNPs or mutated proteins. This interactive process provides an in vivo interactive cellular and molecular diagnostic testing model.

Once the biological sample collection procedure is performed and the data is analyzed, the iMD system proceeds to build a map of the pathology. The iMD is able to detect and build a model of real time chemical fluctuations to analyze patterns in the pathology. In order to assist in the building of complex models, the system employs extensive external computation resources. The combination of limited on-site computing and extensive external computing allows the system to collect and analyze data in real time. This rapid analytical process allows the system to integrate diagnostic and therapeutic elements.

### (II) Fortress Model of IMDs

(2) Implantable IMD that Sends out and Receives Sub-Devices

A single iMD acts as a fortress, sending out probes, analyzing information and providing diagnoses and therapeutics. Since it is implanted, the iMD is typically positioned at the location of tissue pathology. It is therefore able to interact directly in real time with the biological system at the tissue level and the cellular level. In order to collect cell samples, the iMD uses probes and other sub-devices. The probes access specific targeted tissue and collect cell samples, which are then extracted and transported back to the iMD for biopsy. In some cases, the probes interact with a tissue target and obtain information on the status of the tissue, particularly temperature, pressure and other measurable attributes, and transmit the information back to the iMD either with a tethered wire or wireless communication approach.

### (3) IMD Probe Patrol Procedures

In another approach to sending probes to obtain information, the system emits nano-scale probes that move freely in a specific biological sub-system, such as the blood stream, and retrieves the probes in a filter after they make a single pass or multiple passes through the system. Once captured, the cells and molecules in the probes are then transported to the diagnostic LOC facility on the iMD for analysis. In this model of roving probes, the probes are on regular patrols through the system to collect biological samples at specified intervals. The ability to take multiple readings of specific tissues is important to assess the progress of diseases.

The use of probes to tag cells and to track their progress is also a useful model used by iMDs. The iMD then tracks the changes in conditions of a specific collection of targeted cells. This approach is particularly useful in tracking the progress of a dysfunctional protein network in the preparatory phases of analyzing the genetic mutations of cells that generate mutated proteins.

### (4) IMD Active Sensor Array

Rather than have the probes move through the system to obtain information and periodically collect the probes to assess the data, the present invention also employs an active sensor array. In this approach, a network of sensors is fixed in specific tissue locations. The sensor array may be installed by endoscopic surgical techniques or by the iMD itself. The usefulness of a fixed network of sensors is that it provides regular reports of pathology regulation for analysis by the iMD. This provides an on-demand pathology analysis from an assessment of patterns of specific changes in the condition of the targeted tissues.

The sensor array operates by sending signals of changes in the targeted tissues. This is similar to an object moving through an array of cameras in an empty field in which the background is subtracted in order to focus on the changing positions of the object. The iMD obtains the data sets from the sensor array and analyzes the patterns for changes in the condition of the targeted tissue.

### (III) Problem Solving Process of IMDs

### (5) IMD Pathology Problem Identification Process

Genetic pathologies are a form of multi-objective optimization problem (MOOP). In order to identify the source of disease, it is necessary to assess a unique set of combinations of genetic mutations that vary for each individual in slight ways from every other individual. While it is possible to assess the existence of a set of specific genetic mutations, it is necessary to use interaction and testing to identify which of the mutated genes are active in a particular disease. This challenge presents overlapping constraints in the identification of MOOPs among a library of combinatorial options.

The problem solving process begins with the initial assessment of pathology. The system then proposes multiple hypotheses to describe the pathology. The system seeks out cell samples in order to analyze the DNA and protein molecules. In particular, the system seeks to identify specific genetic mutations and protein dysfunctions and the effects of these mutations and dysfunctions on protein regulatory networks. Once the mutated gene combination is identified, the system develops a diagnosis based on the specific composition of genetic mutations and the protein dysfunctions.

The system accesses the results of external microarray analyses and molecular modeling with extensive computer resources. The resulting analyses and modeling provides a map of an individual's unique combination of genetic anomalies. By identifying the sources of pathology, the iMD system is able to develop accurate molecular diagnoses of diseases which lead to precisely targeted cures.

(IV) Mapping Parameters of Pathologies with IMDs
(6) Mapping Pathology Parameters with an IMD for Progressive Diagnostics

A model is built from this process of narrowing the hypotheses of mutated gene combinations that examines the scenarios for the dysfunctional proteins in protein regulatory networks. To identify the molecular mutations, the system accesses libraries of data sets that provide a comparison with healthy genes and proteins. The system then identifies a specific set of dysfunctional genes by focusing on analysis of probable problematic mutated genes and chromosomes by reverse engineering from gene libraries. Precisely by focusing on specific suspect genes, the system is able to produce extremely efficient analyses that refine the hypotheses to narrow the range of affected genes. This process of building models of dysfunctional proteins essentially maps scenarios of the protein regulatory process for each individual within computational constraints.

By using iMDs, this process is dynamic. Continually updated information about the progression of a disease is fed into the iMD for further analysis. This process allows the iMD to develop an evolving map of on-going diagnostics that is provided constantly updated data sets on the actual process of disease evolution as it changes. A model is developed from the data sets that generate scenarios of evolutionary MOOPs (eMOOPs) describing disease progression probabilities. The continual input of data into the iMD system allows the model to be progressively updated until it is able to develop a pathology diagnosis.

When the system is able to obtain only partial information, the system builds an incomplete model that requires specific data sets in order to complete. This partial on-demand genomic analysis focuses on a specific pathology in order to maximize efficiency of limited computation resources. As more information becomes available, for instance through external microarray analyses, then the map of the genetic mutation combinations is made more complete until it is possible to model the parameters of the disease.

From these probable scenarios, the system statistically predicts future disease progression phases with reasonable accuracy.

(7) Developing Vascular maps with IMDs

The human vasculature contains a set of unique proteins that act as markers for specific locations of the cardiovascular system. This view is referred to as “zip codes” for unique vascular tissue locations. The iMD uses this zip code model of vascular locations to identify specific tissues by analyzing specific proteins that match vascular positions.

Once specific proteins are targeted at specific locations, the iMD diagnostic module constructs a map of healthy zip codes (vascular tissue locations). This model allows specific proteins to be tracked through the vasculature. Probes are sent out from the iMDs to track their specific location mobility using this vascular map. Vascular maps are constantly updated as more information is obtained.

Given computation and analytical resource constraints of iMDs, the vascular maps are focused on specific tissue locations with pathologies. Vascular maps are particularly useful because iMDs use mobile probes that require navigation. This mapping approach allows mobile probes to constantly identify their present location until they are either activated to interact with specific tissues or are retrieved by the iMDs for later analyses.

Once a map is generated of specific proteins to specific locations in the vascular system, devices are directed to these precise locations from the iMD in order to provide therapeutics.

### (V) Modeling in IMDs

(8) Modeling Diagnostic Solutions with IMD

After the iMD collects cellular and DNA and protein molecular samples with probes and analyzes the data from the samples, it builds a model using computational resources. The iMD works with multiple iMDs in the distributed network to maximize internal computation resources, but also uses external computer resources for best effect.

The goal of the modeling process is development of a model that describes the source of pathology. By recognizing the modeling challenge as a MOOP with limited constraints, the analysis continually narrows the range of a set of genetic mutations that manifest in disease symptoms.

Since the internal iMD computer network has substantial resource constraints, incomplete models are constructed within time limits to present a putative short-term analysis. While the internal analyses progress, the system uses external computation simultaneously. This parallel use of computation resources allows the on-board computing system to focus on essential modeling construction while the details are identified and solved by the more substantial external computer system.

As more empirical information is presented to the system by continual updates from sensors and probes, the model continues to evolve. This model progression is useful to redirect the probes to obtain more data by collecting more samples from nearby tissue sites. The overall information that is supplied to the models assists the system in mapping complex disease patterns over time. With the new data, not only are the models updated, but the diagnostic algorithms are updated.

Not only are models constructed by collecting and analyzing an individual's cellular and molecular data, but the system compares the model to libraries of healthy and dysfunctional gene and protein models. These comparisons to healthy functional proteins allow the system to model very detailed molecular components. For instance, not only is a dysfunctional protein identified, but the specific nature and character of the mutated protein is identified and modeled. This is important because each deformed protein manifests differently with cell receptors thereby allowing insight in the specificity and type of the dysfunction.

(9) Method of Anticipating Pathologies with Probabilistic Models

While models of individual pathologies are developed and revised with new data, the models are also organized to present multiple scenarios of pathology progression under different contingencies. Each model produces simulations of alternative scenarios within probabilistic constraints.

From these molecular model scenario simulations, the iMD develops anticipatory mechanisms based on the assessment of patterns. These model simulations are valuable because they anticipate behaviors that are useful in therapeutic regimens. For example, when protein markers are identified, they are compared to the model simulations and provide an anticipation of disease progression pathways. The models are also useful in directing the system to look for specific proteins or antibodies in specific locations in order to confirm a diagnosis. As new information on therapeutic feedback is entered into the models, contingency scenario pathways are generated. The process is continually updated. Early analysis and pathology detection allows the system to focus on a specific pathology.

### (VI) Biomarker Detection and Analysis

(10) Biomarker Discovery Procedure with IMD System

One way to detect the presence of a disease is to identify a biomarker. Typically a protein or antibody that represents an adjunct of protein network pathology, discovery of the presence of a biomarker can signify a precursor to a disease state. IMDs are used to collect and detect specific biomarkers. Once a biomarker is identified, the system focuses on collecting quantities of multiple related biomarkers to confirm the existence of a disease and the phase of the disease. After the biomarkers are identified, they are compared to a library of biomarkers to identify the disease progression vector.

An example of biomarker detection and analysis is the identification of the prostate specific antigen (PSA) as a precursor to prostate cancer. However, the iMD analyzes benign and active biomarkers before proposing therapeutic options.

Going one step beyond merely passively identifying biomarkers, the iMDs are used to seek out specific biomarkers. The system efficiently searches for biomarkers that are anticipated by other modeling analyses. Further, the iMDs attract the biomarkers by using as “bait” antibodies and proteins with which they will bind.

### (VII) DNA and Protein Testing

(11) Selective DNA Testing Procedure with IMD System

While specific genetic samples are tested with external DNA microarray (i.e., gene chip) analyses, the present system is useful in analyzing genetic material internally on demand with an iMD. Though relatively limited, the iMD conducts analysis of a small set of gene samples on a specific chromosome. This process begins with the input of DNA samples in the on-board iMD microarray. The system adds analytes to distinguish the genes and passes the resulting data to the diagnostic module for analysis. The results of analysis of a part of a chromosome for gene mutations results in a probabilistic constraint. The system has the advantage of searching for something it is looking for because it starts from the presumption of having knowledge of a disease state with a hypothesis of a probable set of specific gene mutations based on comparisons with gene haplotypes libraries. This model has the advantage of providing a template to the modeling system in order to focus on a specific dysfunction in order to maximize the efficiency of constrained computer resources.

By using the on-board gene chip, the iMD is able to track the accumulation of mutation data over time. By using a combination of modeling and gene testing, the system identifies a matrix of families of unique gene combinations to focus on. This focused search for specific combinations of genes in conjunction with the modeling process allows the iMD system to maximize analytical efficiencies.

From the emerging model that is constructed from selective DNA testing, a typology of disease categories is organized on which to build individualized therapies targeted to specific tissues.

(12) Proteomic Analysis with IMD System

The iMD is used to identify and analyze specific proteins. The iMD diagnostic module accesses the on-board multi-attribute microarray to obtain samples of proteins. The microarray tests proteins by using specific antibodies and proteins as analytes to identify dysfunctional proteins. These dysfunctional proteins anticipate the presence of mutated genes. The model building capabilities of the diagnostic and analytical processes use inference techniques to anticipate the existence of identification of biomarkers. The results of the microarray sampling and detection process are then analyzed by the diagnostic and analytical modules by comparing the dysfunctional proteins to healthy proteins. The diagnostic and analytical modules compare the dysfunctional proteins to a database of proteins that are focused on a specific pathology. This process maximizes the efficiency of constrained computer resources by focusing on a limited subset of probable protein dysfunctions.

In order to maximize computer resources, the iMDs in a network aggregate computational and database operations to solve complex protein combinatorial optimization problems. In more complex cases, such as the analyses of multiple pathologies, the iMDs compare the dysfunctional proteins by using much larger external computer databases.

The analyses of dysfunctional proteins by the analytical and diagnostic iMD modules are critical to development of a model describing the protein regulatory network pathway vectors. A set of mutated genes that generate a combination of dysfunctional proteins cascades a protein regulatory network into disequilibrium. Once a protein regulatory network is modeled, it is possible to develop a set of scenarios that anticipate the existence of dysfunctional proteins. From this model, then, the iMD focuses its search of specific proteins in order to efficiently identify prospective dysfunctional proteins.

If they are detected early, as provided by the iMD system, specific genetic mutations may be localized to specific tissues before they have a more global, and pathological, effect.

### (VIII) External and Tracking Systems

(13) Method for Interaction of IMDs with External Systems

Computational resources on an iMD is constrained. For this reason, it is necessary to use iMD computer resources very efficiently. One way to extend iMD computation capabilities is to aggregate iMDs in a distributed computer network; the group of iMDs share computing functions to maximize resources. However, the modeling of genetic and proteomic optimization problems requires substantial computer resources. For this reason, the iMD network works in conjunction with external computer resources to model and solve complex problems.

In addition to external computer resources, the iMD system works in conjunction with imaging diagnostic systems to create maps of biomedical diseases. However, the advantage of using iMDs is that they are interactive as well as on-site so that they are responsive to the pathology. Therefore, implantable iMDs present the ability to obtain real time data samples, to analyze the data rapidly and to respond immediately to feedback by modifying a diagnosis and therapy. IMDs work in conjunction with external and endoscopic diagnostic approaches.

In one mode of communicating with an external computer system, the iMD automatically sends wireless video signals to a object relational database to monitor disease progression vectors. These data sets are transmitted either wirelessly or by a wire connection at periodic intervals.

Further, by employing external computing systems, the iMD network obtains updated software wirelessly.

(14) Tracking Specific Molecular Regulatory Network Pathologies with IMDs

Traditional medical tracking procedures consist of recording blood pressure, cardiac and neurological electrical patterns, body temperature and other regular bodily functions. The iMD system is used to track more complex protein regulatory network operations. While implantable iMDs are monitoring devices that use probes to collect and analyze cellular and molecular samples and build operational models of disease physiological states, they are also useful in actively regulating functionality. The iMD system is used to focus on particular pathologies in protein regulatory networks. This allows the iMDs to monitor treatment as well.

While the system possesses a passive tracking and monitoring mechanism to record and store data about pathology progress, it is also active in providing an interactive mechanism for changing the disease progression by supplying remedies.

Multiple iMDs are organized to track a specific disease progression by sharing sensor and probe data sets. The data is then analyzed by multiple iMDs to construct a unified model. The model is compared to libraries of functional proteins and healthy protein regulatory networks in order to anticipate and track specific dysfunctional proteins that generate from mutated genes.

When the iMD system provides therapeutic remedies to correct specific proteins, they diagnostic system tracks, records and stores data about these remedies. As the disease evolves, the iMD system constantly interacts with the proteins by providing, assessing and refining the remedies.

Reference to the remaining portions of the specification, including the drawings and claims, will realize other features and advantages of the present invention. Further features and advantages of the present invention, as well as the structure and operation of various embodiments of the present invention, are described in detail below with respect to accompanying drawings.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes in their entirety.

### DESCRIPTION OF THE DRAWINGS

FIG. 1 is a schematic diagram showing the extraction of cellular and molecular samples to an iMD with probes.

FIG. 2 is a schematic drawing of an iMD sending and receiving a probe to obtain a sample and simultaneously extracting samples.

FIG. 3 is a schematic diagram showing a diagnostic module of an iMD sending a probe to tissue and sending back data wirelessly.

FIG. 4 is a flow chart showing the process of an iMD retrieving and analyzing data from a probe.

FIG. 5 is a schematic diagram showing the order sequence of a process of obtaining and analyzing diagnostic data and applying therapy using an iMD.

FIG. 6 is a schematic diagram showing the process of an iMD sending and receiving probes at a later location.

FIG. 7 is a flow chart showing the process of an iMD sending and tracking probes and analyzing their data.

FIG. 8 is a schematic diagram showing an iMD interacting in a specific sequence with a tissue with an embedded sensor array.

FIG. 9 is a flow chart showing the process of analyzing pathology by using the analytical and diagnostic modules of an iMD.

FIG. 10 is a flow chart showing the analytical process of the iMD's analytical module to solve problems.

FIG. 11 is a schematic diagram showing the process of developing an updated model by using parallel data samples in two diagnostic modules of an iMD.

FIG. 12 is a flow chart showing the process of updating a model in an iMD analytical module with updated data sets from the diagnostic module.

FIG. 13 is a drawing of vascular zip codes that identify specific locations in the vasculature.

FIG. 14 is a flow chart showing the process of analyzing data to map and track vascular locations with probes by coordinating the diagnostic and analytical modules of an iMD.

FIG. 15 is a flow chart showing the process of collecting and analyzing patient data using diagnostic and analytical modules of an iMD.

FIG. 16 is a flow chart showing the process of developing therapeutic options using the diagnostic and analytical modules of an iMD.

FIG. 17 is a flow chart showing the process of collecting and analyzing biomarkers using the diagnostic and analytical modules of an iMD.

FIG. 18 is a flow chart showing the process of analyzing DNA data, including gene mutation data, using the diagnostic and therapeutic modules of an iMD.

FIG. 19 is a flow chart showing the process of analyzing samples in a microarray in the diagnostic module and analyzing and modeling the data in the analytical module of an iMD.

FIG. 20 is a schematic diagram showing a network of four iMDs integrated with external computation.

FIG. 21 is a schematic diagram describing the coordination of an iMD network with an imaging diagnostic system.

FIG. 22 is a flow chart showing the process of tracking and managing pathology using the analytical and diagnostic modules of an iMD.

### DETAILED DESCRIPTION OF THE DRAWINGS

The key to solving medical pathologies is to accurately diagnose the problem. Diagnosis involves some detective work. Problem finding involves multivariate analysis to decipher complex disorders. In other cases, such as cancer, which has a genetic component that causes dysfunctional protein combinations to distort cellular networks, a multitude of genetic mutations require analysis before an accurate diagnosis is possible.

The diagnostic module of the iMD, in combination with the analytical module and the therapeutic module, is well suited to analyzing cell and molecular samples in order to identify the source of pathologies. Cell, DNA and protein samples are input into the diagnostic module, tested, analyzed and modeled. Solution options are then generated from the model. In some cases, the complexity of the pathology, or pathologies, require an experimentation process which involves testing variables and receiving feedback to narrow the range of causes.

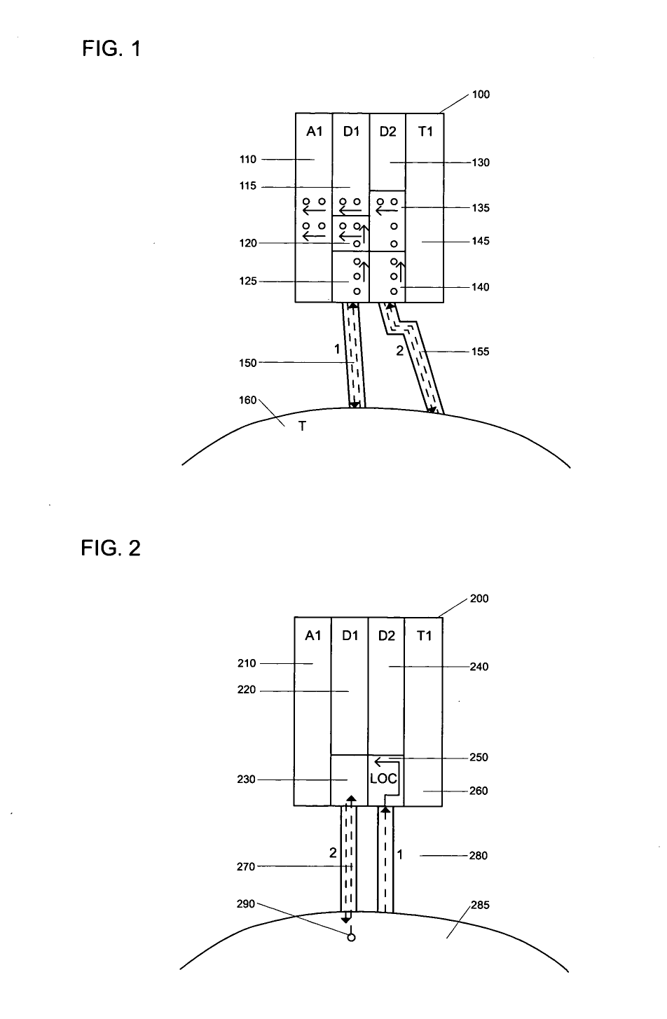

FIG. 1 shows an iMD (100) with two diagnostic modules (115 and 130), an analytical module (110) and a therapeutic module (145). The diagnostic modules access the external environment with probes which are accessible by attached tubes. The probes are launched into specific tissue (160), collect biological and molecular samples (150 and 155) and extract the samples from the tissue. The samples are then initially input into a chamber of each diagnostic module (125 and 140) and moved to inner chambers (120 and 135) for organization and analysis. The samples are then analyzed by a μTAS or a LOC, with the data results sent to the analytical module. This process is performed in parallel by the two diagnostic modules, which are able to collect and analyze samples from different tissues simultaneously. The μTAS may be a microarray or a microfluidic device for analysis of DNA, RNA, whole cell or proteins. Whole cell analysis involves cell trapping, cell sorting, cell treatment and cell analysis phases in a sequence. Further, the μTAS may contain radial components in some layers. The μTAS and LOC devices also may contain reverse-phase or forward-phase protein microarrays.

FIG. 2 shows the iMD sending a probe from and receiving a probe back to one diagnostic module and receiving the probe back to another diagnostic module. The iMD (200) consists of an analytical module (210), diagnostic module 1 (220), diagnostic module 2 (240) and a therapeutic module (260). Diagnostic module 2 receives a cell sample and analyzes the sample in the LOC. Diagnostic module 1 then sends a probe to the cell cluster position at 290 in the tissue (285) and receives back the probe with cell samples. The first cell sample orients the system, while the second diagnostic module is able to collect precise samples.

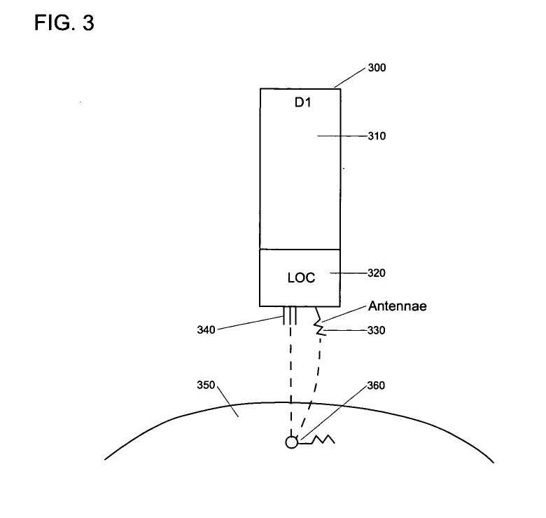

In FIG. 3, the process of transmitting data wireless to the diagnostic module is shown. In this case, a probe (360) is sent to a cell cluster (350) from the diagnostic module (300). An antennae (330) receives the signal from the probe and transmits the data for analysis.

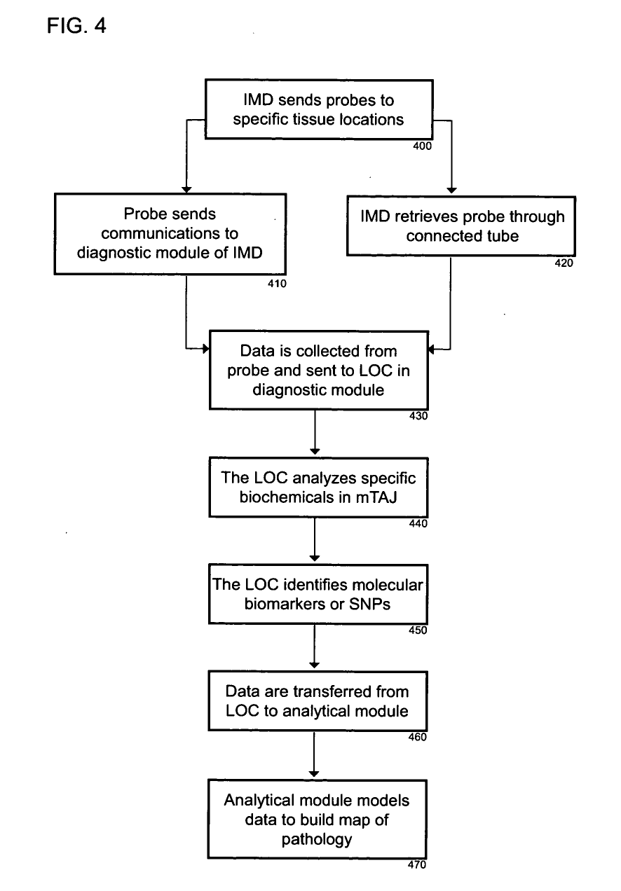

FIG. 4 shows the process of an iMD retrieving and analyzing data from a probe. After the iMD sends probes to specific tissue locations (400), each probe sends communications to a diagnostic module (410). The iMD retrieves the probes through connected tubes (420) and data is collected from the probes and sent to the LOC in the diagnostic module (430). The LOC analyzes specific biochemicals (440) and identifies molecular biomarkers or SNPs (450). The data are transferred from the LOC to the analytical module (460) and the analytical module models data to build a map of the pathology (470).

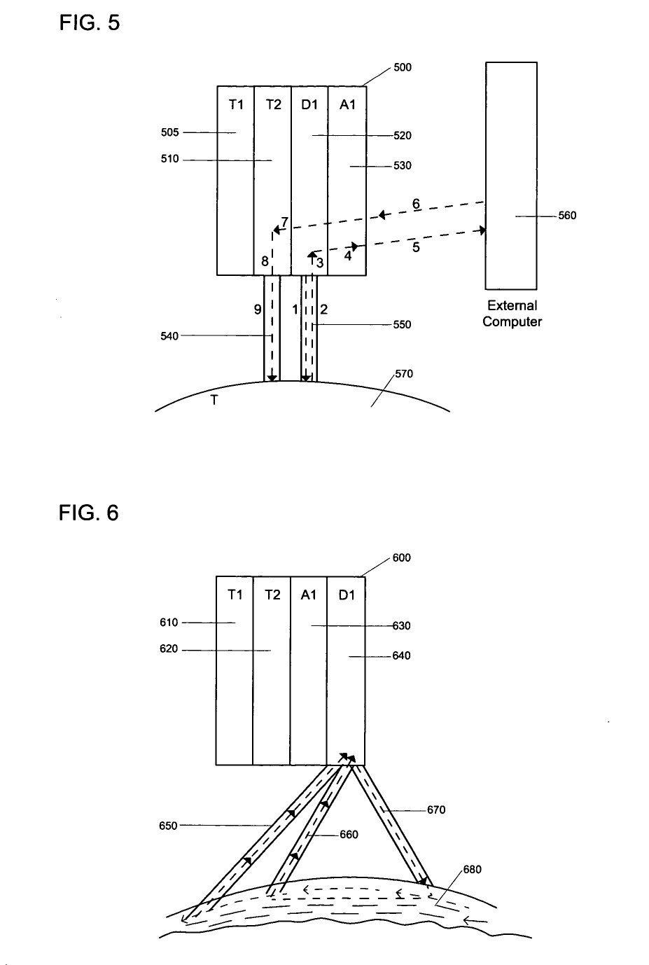

FIG. 5 shows the order sequence of a process of obtaining and analyzing diagnostic data and applying therapy using an iMD. The iMD (500) is configured with two therapeutic modules (505 and 510), a diagnostic module and (520) and an analytical module (530). After first sending probes (550) from the diagnostic module, the diagnostic module receives the probes. After analyzing the data in an LOC or μTAS, the diagnostic module then sends the data to the analytical module and to the external computer (560) for analysis and modeling of the data. The external computer sends modeling data back to the analytical module, which then forwards solution options to the therapeutic module. The therapeutic module then combines biologicals and chemicals and provides a therapy (540) to the tissue (570).

FIG. 6 shows the process of an iMD sending and receiving probes at a later position. The diagnostic module (640) first sends probes (670) to a region. The probes move position and are collected at new locations (650 and 660) for analysis.

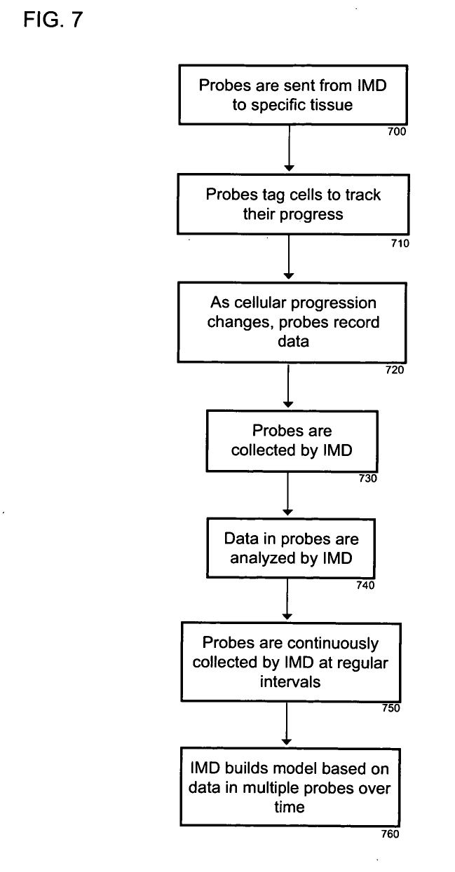

FIG. 7 shows the process of an iMD sending and tracking probes and analyzing their data. Once the probes are sent from an iMD to specific tissue (700), probes tag cells to track their progress (710). As cellular progression changes, the probes record data (720) and are collected by the iMD (730). The data in the probes are analyzed by the iMD (740) and the probes are continuously collected by the iMD at regular intervals (750). While the data in the probes are analyzed, and continuously collected, the iMD builds a model based on the data in multiple probes over time (760).

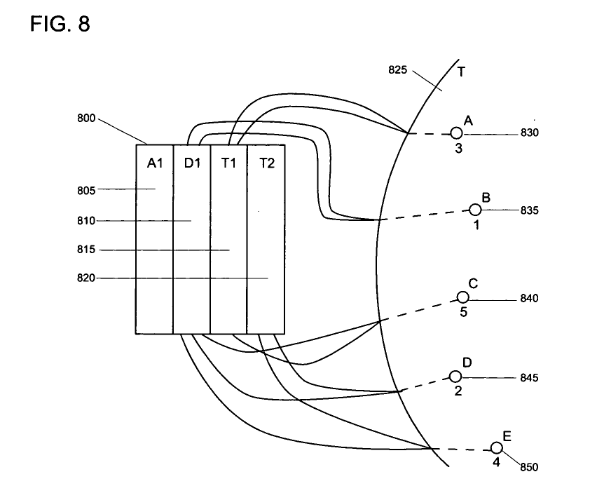

FIG. 8 shows an iMD interacting in a specific sequence with a tissue with an embedded sensor array. The sensor array is noted at 830-850. The sensors send data to the diagnostic module (810) and the therapeutic modules (815 and 820) at regular intervals. In the example shown, sensor B (835) sends data to the diagnostic module first. Then, sensor D (845) sends data to the diagnostic module. Next, sensor A (830) sends data to the diagnostic module, followed by sensor E (850) and finally sensor C (840). The therapeutic modules send therapies to the locations of the sensors in the order of priorities.

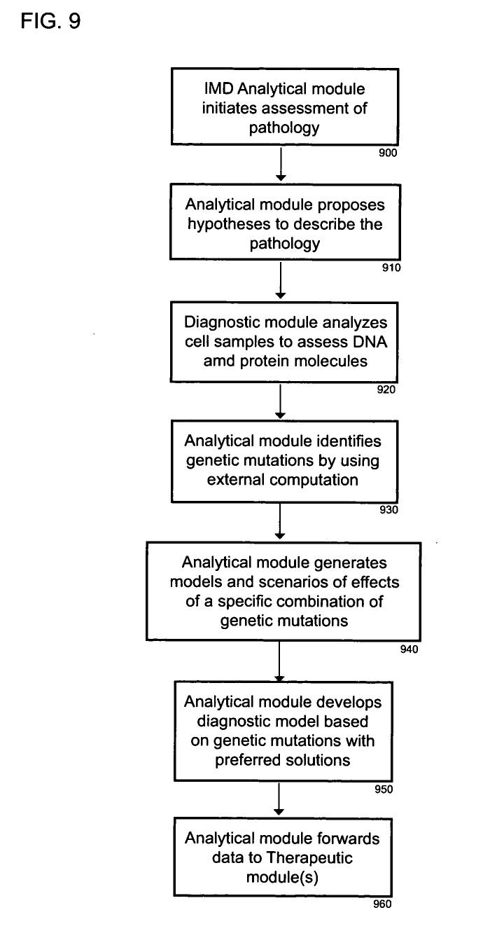

FIG. 9 shows the process of analyzing pathology by using the analytical and diagnostic modules of an iMD. The iMD analytical module first initiates an assessment of pathology (900) and proposes hypotheses to describe the pathology (910). The diagnostic module then analyzes cell samples to assess DNA and protein molecules (920). The analytical module identifies genetic mutations by using external computation (930) and generates models and scenarios of effects of a specific combination of genetic mutations (940). The analytical module then develops a diagnostic model based on genetic mutations with a preferred solution (950) and forwards the data to the therapeutic module(s) (960).

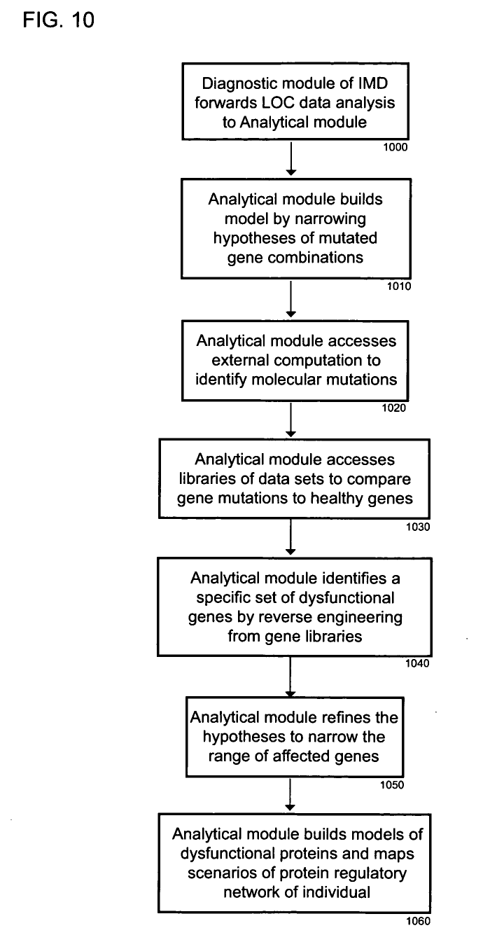

FIG. 10 shows the analytical process of the iMD's analytical module to solve problems. After the diagnostic module of the iMD forwards LOC data analysis to the analytical module (1000), the analytical module builds a model by narrowing hypotheses of mutated gene combinations (1010). The analytical module accesses external computation to identify molecular mutations (1020) and then accesses libraries of data sets to compare gene mutations to healthy genes (1030). The analytical module identifies a specific set of dysfunctional genes by reverse engineering from gene libraries (1040) and refines the hypotheses to narrow the range of affected genes (1050). The analytical module then builds models of dysfunctional proteins and maps scenarios of protein regulatory network of an individual (1060). In one embodiment of the invention, the analytical functions are integrated into the therapeutic module. In another embodiment, the analytical functions are integrated into the diagnostic module.

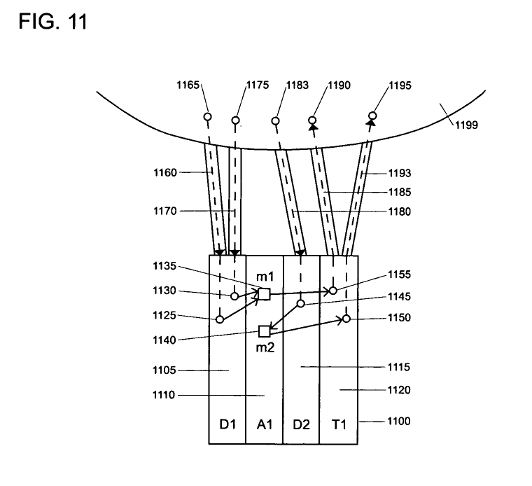

FIG. 11 shows the process of developing an updated model by using parallel data samples in two diagnostic modules of an iMD. Diagnostic module 1 (1105) receives two sets of samples (1160 and 1170) from tissue (1199) at cell clusters (1165 and 1175) and diagnostic module 2 (1115) receives a set of samples (1180) from another cell cluster (1183). The diagnostic modules analyze the cell samples (1125, 1130 and 1145) and transfer the data to the analytical module (1110). The analytical module builds two models. The first model (1135) is based on data from the samples from diagnostic module one, while the second model (1140) is based on data from samples from diagnostic module two. The first model develops solution options and forwards the data to the therapeutic module (1155), while the second model develops solutions and forwards the data to the therapeutic module (1150). In one embodiment, there are two or more separate therapeutic modules that accept different model solutions to solve different pathologies simultaneously. The first model solutions are constructed and sent (1185) to a cell site (1190) near the original cell clusters. The second model solutions are constructed and sent (1193) to the a cell site (1195) in the target tissue (1199).

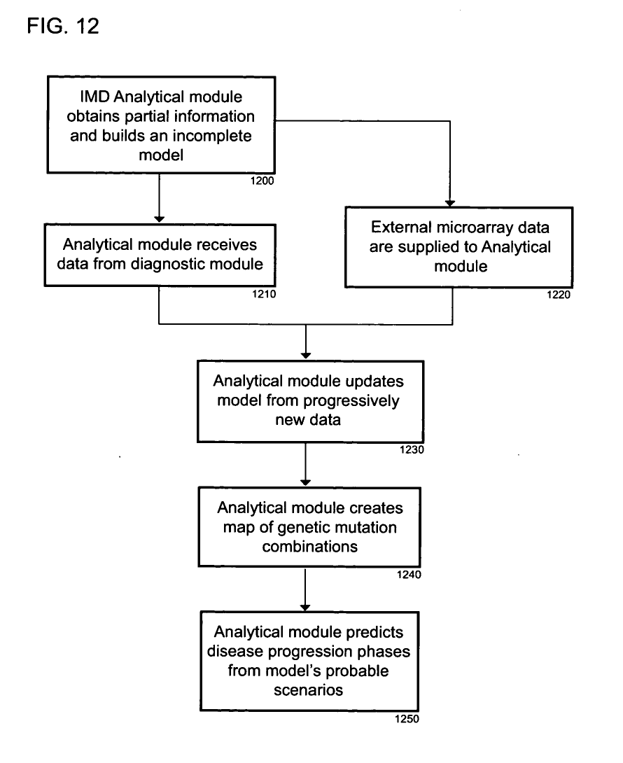

FIG. 12 shows the process of updating a model in an iMD analytical module with updated data sets from the diagnostic module. After the iMD analytical module obtains partial information and builds an incomplete model (1200), the analytical module receives data from the diagnostic module (1210) as well as external microarray data (1220). The analytical module updates the model from progressively new data (1230) and creates a map of genetic mutation combinations (1240). The analytical module then predicts disease progression phases from model's probable scenarios (1250).

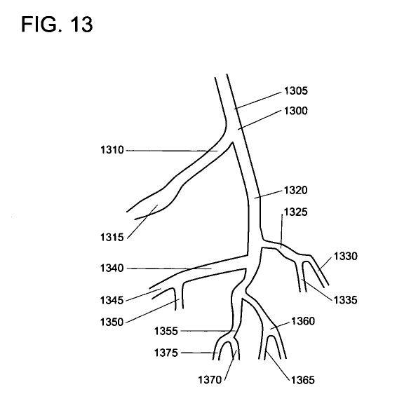

FIG. 13 shows the vascular zip codes that identify specific locations in the vasculature. Each location (1305-1375) represents a distinctive location that is recognized by a specific set of proteins.

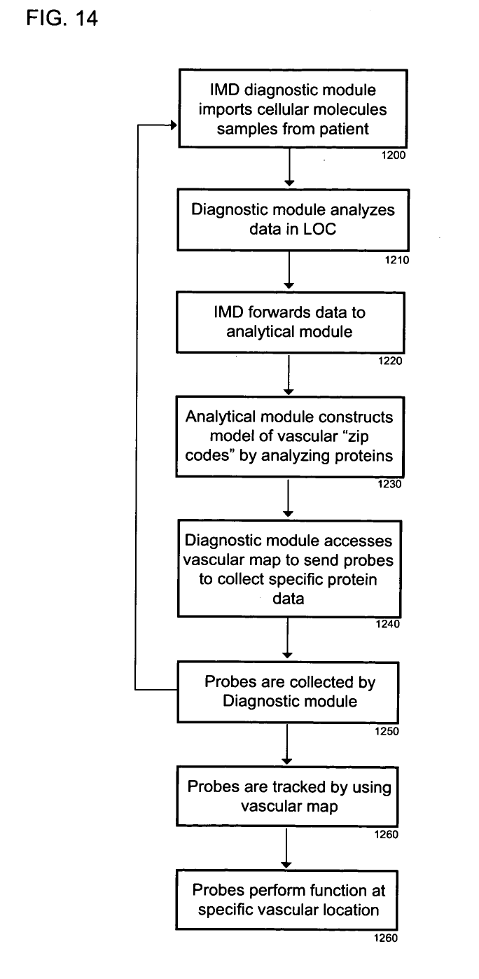

FIG. 14 shows the process of analyzing data to map and track vascular locations with probes by coordinating the diagnostic and analytical modules of an iMD. Once the iMD diagnostic module imports cellular and molecular samples from a patient (1400), the diagnostic module analyzes the data in its LOC (1410) and forwards the data to the analytical module (1420). The analytical module constructs a model of vascular “zip codes” by analyzing proteins (1430) and the diagnostic module accesses the vascular map to send probes to collect specific protein data (1440). The probes are collected by the diagnostic module (1450) and are tracked by using the vascular map (1460). Once they arrive at specific targeted locations in the vasculature, the probes perform a function at a specific vascular location (1470).

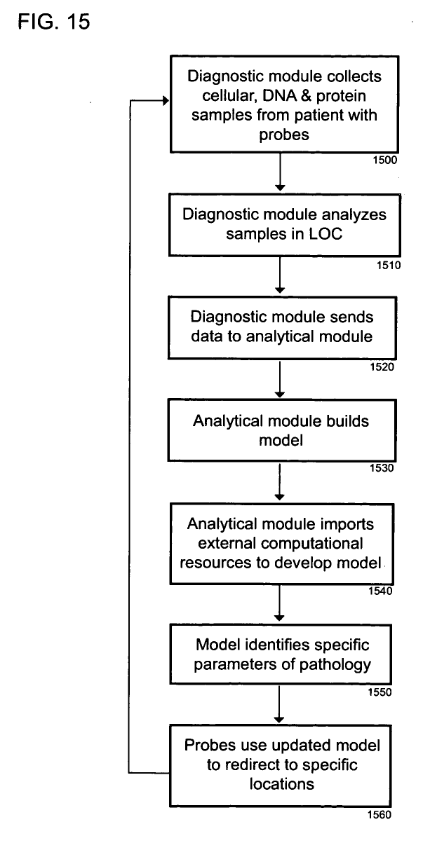

FIG. 15 shows the process of collecting and analyzing patient data using diagnostic and analytical modules of an iMD. The diagnostic module first collects cellular, DNA and protein samples from a patient with probes (1500) and then analyzes the samples in the LOC (1510). The diagnostic module sends data to the analytical module (1520), which builds a model (1530) and imports external computational resources to develop the model (1540). The model identifies specific parameters of pathology (1550). Probes then use updated versions of the model to redirect to specific locations (1560).

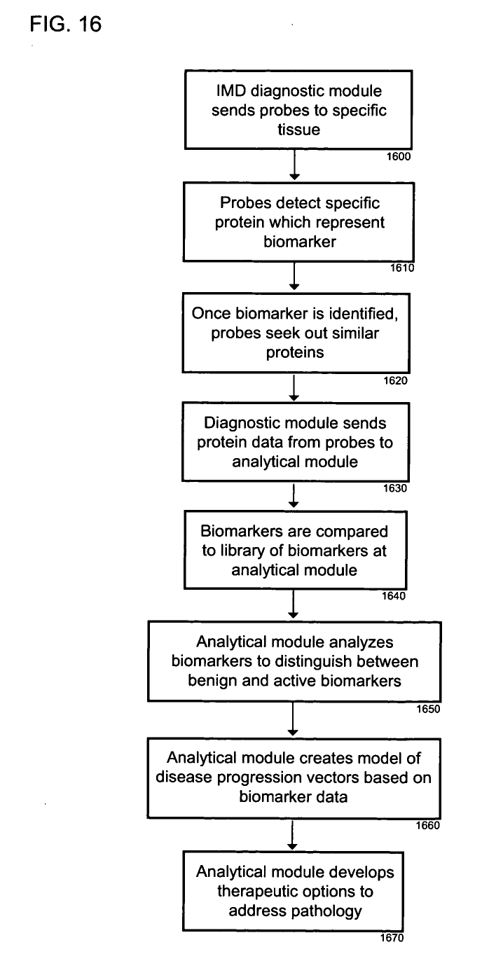

FIG. 16 shows the process of developing therapeutic options using the diagnostic and analytical modules of an iMD. After the iMD diagnostic module sends probes to specific tissue (1600), the probes detect specific protein which represent a biomarker (1610). Once a biomarker is identified, probes seek out similar proteins (1620) and the diagnostic module sends protein data from probes to the analytical module (1630). The biomarkers are compared to a library of biomarkers at the analytical module ((1640), which analyzes biomarkers to distinguish between benign and active biomarkers (1650).

The analytical module creates a model of disease progression vectors based on biomarker data (1660) and develops therapeutic options to address pathology (1670).

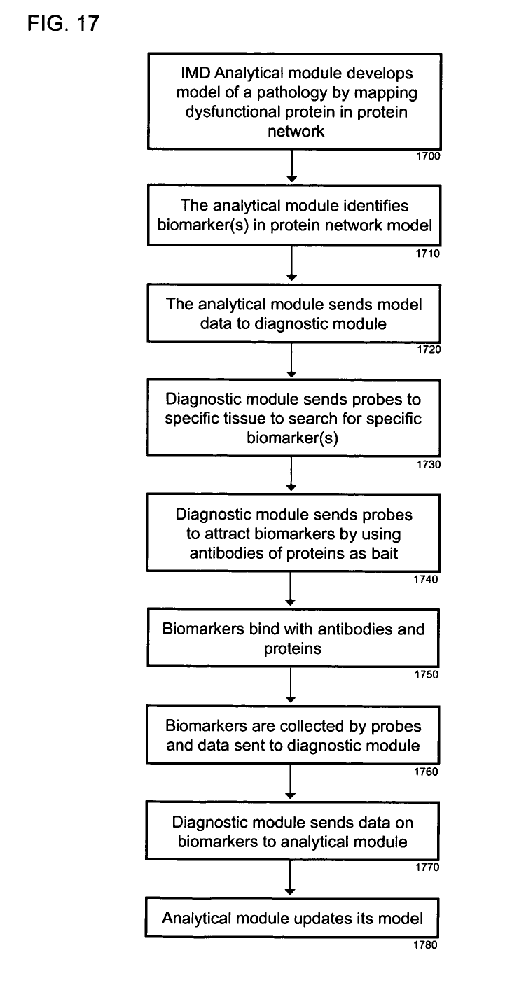

FIG. 17 shows the process of collecting and analyzing biomarkers using the diagnostic and analytical modules of an iMD. First, the analytical module develops a model of pathology by mapping dysfunctional protein in a protein network (1700). The analytical module then identifies biomarker(s) in a protein network model (1710) and sends model data to the diagnostic module (1720). Next, the diagnostic module sends probes to specific tissue to search for specific biomarker(s) (1730) and sends probes to attract biomarkers by using antibodies or proteins as bait (1740). Biomarkers bind with antibodies and proteins (1750) and are collected by probes and data are sent to the diagnostic module (1760). The diagnostic module then sends data on biomarkers to the analytical module (1770), which updates its model (1780). In another embodiment, two or more diagnostic modules are used for this process and the modules divide tasks to assess two or more different pathologies. In this case, the analytical module receives data from and sends data to the two or more diagnostic modules.

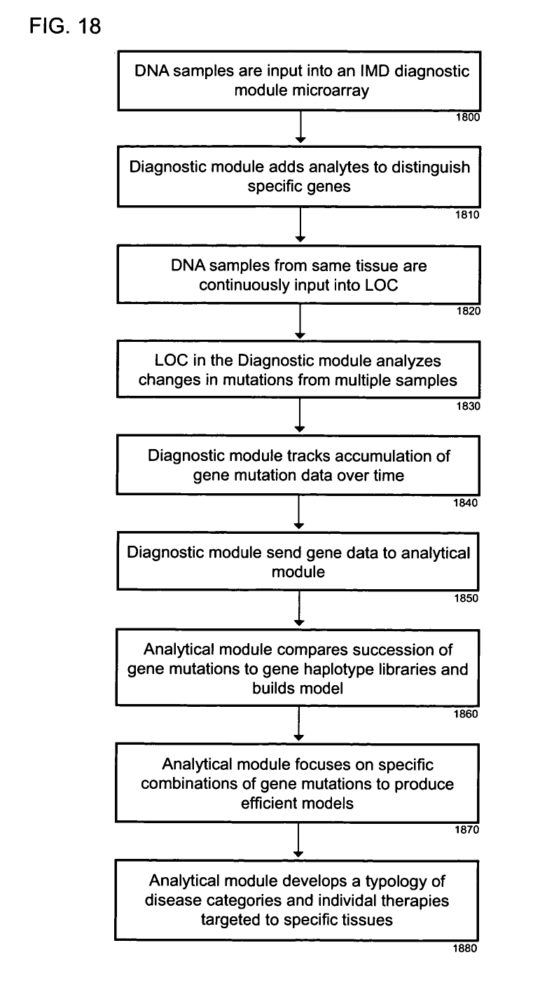

FIG. 18 shows the process of analyzing DNA data, including gene mutation data, using the diagnostic and therapeutic modules of an iMD. DNA samples are initially input into an iMD diagnostic module microarray (1800). The diagnostic module adds analytes to distinguish specific genes (1810). The DNA samples from some tissue are continuously input into the diagnostic module LOC (1820) and the LOC analyzes changes in mutations from multiple samples (1830). The diagnostic module tracks the accumulation of gene mutation data over time (1840) and sends the gene data to the analytical module (1850). The analytical module compares the succession of gene mutations to gene haplotypes libraries and builds a model (1860). The analytical module focuses on specific combinations of gene mutations to produce efficient versions of models (1870). The analytical module then develops a typology of disease categories and individual therapies targeted to specific tissues (1880).

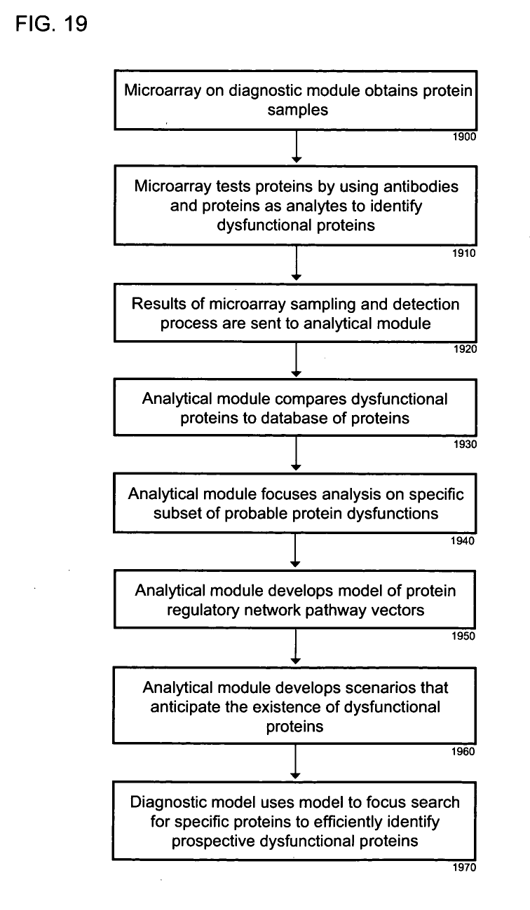

FIG. 19 shows the process of analyzing samples in a microarray in the diagnostic module and analyzing and modeling the data in the analytical module of the iMD. The microarray on the diagnostic module first obtains protein samples (1900) and then tests the proteins by using antibodies and proteins as analytes to identify dysfunctional proteins (1910). The results of microarray sampling and detection processes are sent to the analytical module (1920), which compares dysfunctional proteins to a database of proteins (1930). The analytical module focuses analysis on a specific subset of probable protein dysfunctions (1940) and develops a model of protein regulatory network pathway vectors (1950). The analytical module develops scenarios that anticipate the existence of dysfunctional proteins (1960) and the diagnostic module then uses the model to focus a search for specific proteins to efficiently identify prospective dysfunctional proteins (1970).

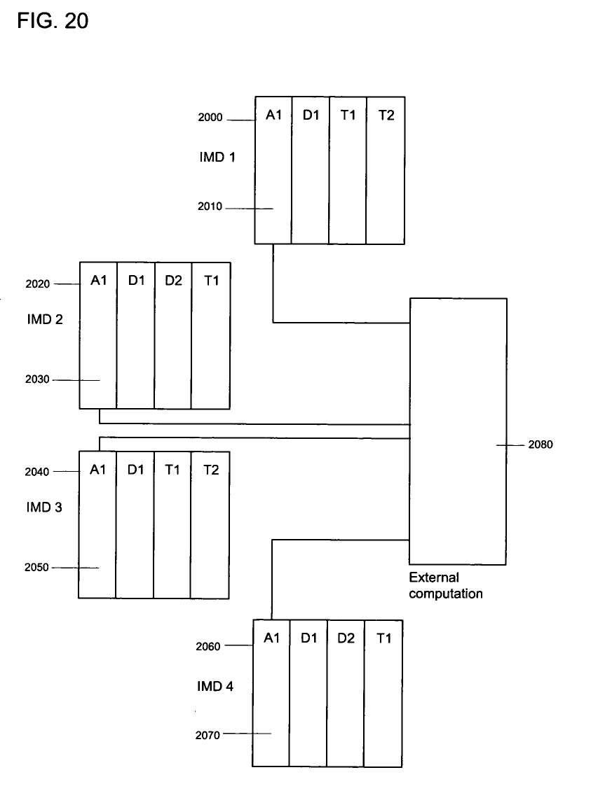

FIG. 20 shows a network of four iMDs integrated with external computation. The iMDs (2000, 2020, 2040 and 2060) are each attached to external computation (2080) at the analytical modules (2010, 2030, 2050 and 2070).

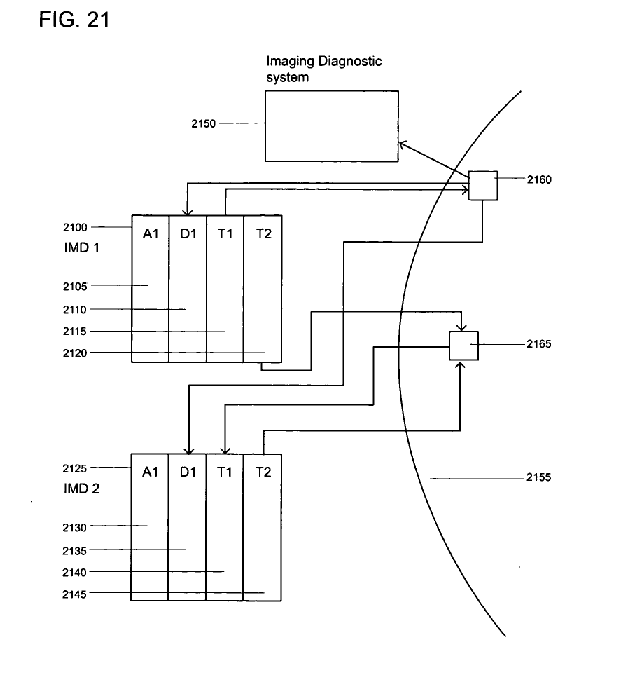

FIG. 21 shows the coordination of an iMD network with an imaging diagnostic system. The imaging system (2150) develops an initial diagnosis of a cell cluster (2160) of specific pathology to guide the iMD system. IMD 1 (2100) sends probes that provide samples to D1 (2110) and the iMD 2 D1 (2135), which supplies information to A1 (2105) for modeling and solution options are sent to T1 (2115) for therapy application to the cell cluster (2160). Another cell cluster pathology is identified (2165) in the tissue (2155) and iMD 2 (2125) receives samples at D1 (2140), analyzes the samples and supplies data to the analytical module for modeling. Solution options are then sent to T1 (2145), which applies a therapy to the cell cluster (2165). The two iMDs work together to simultaneously solve two pathologies. In one embodiment of the system, the external imaging system is a functional MRI. The combination of a f-MRI with the iMD system provides a powerful set of dynamic tools for effective treatment.

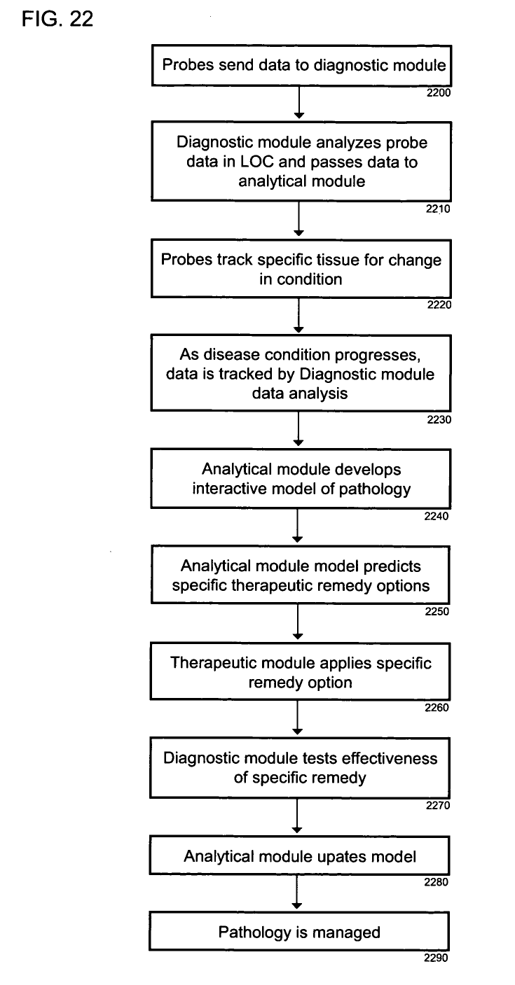

FIG. 22 shows the process of tracking and managing pathology using the analytical and diagnostic modules of an iMD. After the probes send data to the diagnostic module (2200), the diagnostic module analyzes probe data in the LOC and passes the data to the analytical module (2210). The probes track specific tissue for change in condition (2220) and, as a disease condition progresses, data is tracked by the diagnostic module data analysis (2230). The analytical module develops an interactive model of pathology (2240) and the model predicts specific therapeutic remedy options (2250). The therapeutic module applies a specific remedy option (2260) and the diagnostic module tests the effectiveness of the specific remedy (2270). As the therapeutic module applies refined remedies, the process repeats. The analytical module updates the model (2280) and the pathology is managed (2290).

I claim:

1. A system for operation of a medical device for diagnostics, comprising:
a set of layers of medical device components;
a set of electrical interconnects;
a set of microfluidic components, including tubes, valves and gates;
at least one integrated circuit;
wherein the medical device components include a lab-on-a-chip (LOC) device;
wherein the medical device components include a micro-total analysis systems (μTAS) device;
wherein the medical device components are connected by electrical interconnects;
wherein the medical device components are connected by microfluidic components;
wherein the medical device components are on layers of a multi-layer apparatus;
wherein the medical device consists of at least two layers;
wherein when cell, gene, protein or RNA samples are input into the LOC or μTAS, the samples are tested by applying analytes and receiving a resultant feedback;
wherein the test result is analyzed by the integrated circuit and stored in memory; and
wherein the data from the diagnosis is forwarded to an analytical module of a medical device for modeling.

2. A system for operation of a medical device for diagnostics, comprising:
a set of layers of medical device components;
a set of electrical interconnects;
a set of microfluidic components, including tubes, valves and gates;
at least one integrated circuit;
wherein the medical device components include at least two lab-on-a-chip (LOC) devices;
wherein each LOC receives cell, gene, protein and RNA samples;
wherein each LOC assesses the type of sample the other is testing and coordinates the samples to increase the efficiency of the timing of the testing process;
wherein each LOC adds one of a set of analytes to analyze the samples;
wherein each LOC receives data from the result of the test of the analytes on the samples; and
wherein each LOC transmits the test data results to at least one analytical module in the medical device.
